(19) United States
(12) Patent Application Publication
(10) Pub. No.: US 2026/0267626 A1
(43) Pub. Date: Sep. 10, 2026

(54) METHOD TO ANALYZE SOURCE DATA GENERATED BY ONE OR MORE BIOPROCESSING DEVICE

(71) Applicant: Cytiva Sweden AB, Uppsala (SE)

(72) Inventors: Key Hyckenberg, Uppsala (SE); Olof Nergård, Uppsala (SE); Jens Widehammar, Uppsala (SE); Rickard Adolfsson, Uppsala (SE)

(21) Appl. No.: 19/165,515

(22) PCT Filed: Mar. 4, 2024
(86) PCT No.: PCT/EP2024/055537

§ 371 Date: Sep. 15, 2025

(30) Foreign Application Priority Data
Mar. 15, 2023 (SE) 2350288-3

(51) Int. Cl. G06F 8/41; G01N 30/86

(52) U.S. Cl. CPC G06F 8/447; G01N 30/8696

(57) ABSTRACT

In one aspect, the present invention relates to computer implemented method (800) performed by an analytics module (630) configured to analyze source data generated by one or more bioprocessing device (100). The method comprises sending (810) a request (631) comprising identifier in the form of source paths, receiving (820) a response (622) comprising source data, and analyzing (830) the response (622) to generate target paths and target data, wherein analyzing comprises executing a script, and sending a message comprises the target paths (632) and/or the target data (633).

### TECHNICAL FIELD

The present invention relates to a method for analyzing source data generated by one or more bioprocessing device.

### BACKGROUND

Bioprocessing systems are widely used, e.g. to perform biomolecule/protein separation. An example of a bioprocessing system is a chromatography system. Chromatography is a well-known procedure for purifying protein samples. The sample may typically be provided in a fluid, e.g. deriving from a bioreactor.

A bioprocessing system/device is generally used to provide a particular system functionality, e.g., the bioprocessing system/device may be used to produce and/or separate a desired substance, e.g., protein purification in a bioprocess such as chromatography or filtration or production through cell cultivation or oligo synthesis.

During such separation/processing large amounts of data is collected during elution etc. of samples. Further, additional data related to the elution of samples etc. may be collected, such as characteristics of the surrounding environment (e.g. temperature) when performing chromatography, cell culture, filtration, or synthesis.

In one example, data from multiple runs or data from multiple chromatography apparatuses are collected and aggregated to improve quality of a final result of analysis. This increases complexity when aggregating data, at least partially due to various phenomenon such as baseline drift, changes in the shape of elution peaks, and shifts in the elution times.

Conventional solutions of chromatography analysis systems etc. typically load all raw data from the multiple chromatography/device runs and attempt to derive aggregated data. This has the drawback that only a selected part of the multiple chromatography/device runs can be analyzed at one given moment due to constraints of memory and processing resources of the analysis systems.

Thus, there is a need for an improved method for analyzing source data derived from bioprocessing devices.

### OBJECTS OF THE INVENTION

An objective of embodiments of the present invention is to provide a solution which mitigates or solves the drawbacks and problems described above.

### SUMMARY OF THE INVENTION

The above and further objectives are achieved by the subject matter described herein.

Further advantageous implementation forms of the invention are further defined herein.

According to a first aspect of the invention, the above mentioned and other objectives are achieved by a computer implemented method performed by an analytics module configured to analyze source data generated by one or more bioprocessing devices.

The method comprises sending a request comprising identifier in the form of source paths, receiving a response comprising source data, analyzing the response to generate target paths and target data, wherein analyzing comprises executing a script, and sending a message comprising the target paths and the target data.

An advantage of embodiments according to the first aspect is that complexity of performing analysis of source data generated by one or more bioprocessing devices can be reduced at the same time as flexibility of analysis of source data generated by one or more bioprocessing devices is increased.

In one embodiment of the first aspect, the source data is indicative of points of interests or subsets of data derived from the source data.

In one embodiment of the first aspect, the script comprises a selection of any of instructions defining a flow of execution, operations defining an algorithm that generates the target data using the source data and rules defining if an operation should be executed or not using execution context.

According to a second aspect of the invention, the above mentioned and other objectives are achieved by a computer implemented method performed by a source module configured to use source data generated by one or more bioprocessing device, the method comprising receiving a first request comprising an identifier in the form of source paths, sending a second request using a resolver indicated by the source paths, receiving a response comprising a subset of raw data, generating source data using the subset of raw data, sending a response comprising the source data.

According to a third aspect of the invention, the above mentioned and other objectives are achieved by a computer implemented method performed by a target module configured to output target data using target paths, the method comprising receiving a message comprising the target paths and the target data, sending a first message to store the target data using a resolver indicated by the target paths, or sending a second message to display the target data using a resolver indicated by the target paths.

According to a fourth aspect of the invention, the above mentioned and other objectives are achieved by a computer comprising a processor, and a memory, said memory containing instructions executable by said processor, wherein said computer is configured to perform the method according to any of the first, second or third aspect.

According to a fifth aspect of the invention, the above mentioned and other objectives are achieved by a computer program comprising computer-executable instructions for causing a computer, when the computer-executable instructions are executed on a processing unit comprised in the computer, to perform any of the method steps according to any of the first, second or third aspect.

According to a sixth aspect of the invention, the above mentioned and other objectives are achieved by a computer program product comprising a computer-readable storage medium, the computer-readable storage medium having the computer program according to the fifth aspect stored therein.

According to a seventh aspect of the invention, the above mentioned and other objectives are achieved by a bioprocessing support system configured to analyze source data, the method comprising sending, by an analytics module, a request comprising identifier in the form of source paths, receiving, by a source module, the first request comprising an identifier in the form of source paths, sending, by the source module, a second request using a first resolver indicated by the source paths, receiving, by the source module, a first response comprising a subset of raw data, generating, by the source module, source data using the subset of raw data, sending, by the source module, a second response comprising the source data, receiving, by the analytics module, the second response comprising the source data, analyzing, by the analytics module, the second response to generate target paths and target data, wherein analyzing comprises executing a script, and sending, by the analytics module, a first message comprising the target paths and the target data.

In one embodiment of the seventh aspect, the method further comprises receiving, by a target module, the first message comprising the target paths and the target data, sending, by the target module, a second message to store the target data using a resolver indicated by the target paths, or sending, by the target module, a third message to display the target data using a resolver indicated by the target paths.

Advantages of the second to seventh aspects are at least the same as for the first aspect.

Further applications and advantages of embodiments of the invention will be apparent from the following detailed description.

### BRIEF DESCRIPTION OF THE DRAWINGS

FIG. 1 shows a chromatography system embodied as a chromatography apparatus according to one or more embodiments of the disclosure.

FIG. 2A illustrates an example of result data generated by one or more chromatography devices according to one or more embodiments of the disclosure.

FIG. 2B illustrates deriving of points of interest according to one or more embodiments of the disclosure.

FIG. 3 illustrates an operation generating target data according to one or more embodiments of the disclosure.

FIG. 4 illustrates raw result data generated from a plurality of chromatography systems according to one or more embodiments of the disclosure.

FIG. 5 illustrates raw result data generated at different chromatography runs according to one or more embodiments of the disclosure.

FIG. 6 illustrates flow of data between functional modules of a computer performing a method according to one or more embodiments of the present disclosure.

FIG. 7 shows a computer according to one or more embodiments of the present disclosure.

FIG. 8 shows a flowchart of a method according to one or more embodiments of the present disclosure.

FIG. 9 shows a flowchart of a method according to one or more embodiments of the present disclosure.

FIG. 10 shows a flowchart of a method according to one or more embodiments of the present disclosure.

FIG. 11 shows a high-level practical workflow that may be used in various embodiments of the invention.

FIG. 12 shows a more detailed embodiment of template execution for generating desired end data.

A more complete understanding of embodiments of the invention will be afforded to those skilled in the art, as well as a realization of additional advantages thereof, by a consideration of the following detailed description of one or more embodiments. It should be appreciated that like reference numerals are used to identify like elements illustrated in one or more of the figures.

### DETAILED DESCRIPTION

An “or” in this description and the corresponding claims is to be understood as a mathematical OR which covers “and” and “or”, and is not to be understand as an XOR (exclusive OR). The indefinite article “a” in this disclosure and claims is not limited to “one” and can also be understood as “one or more”, i.e., plural.

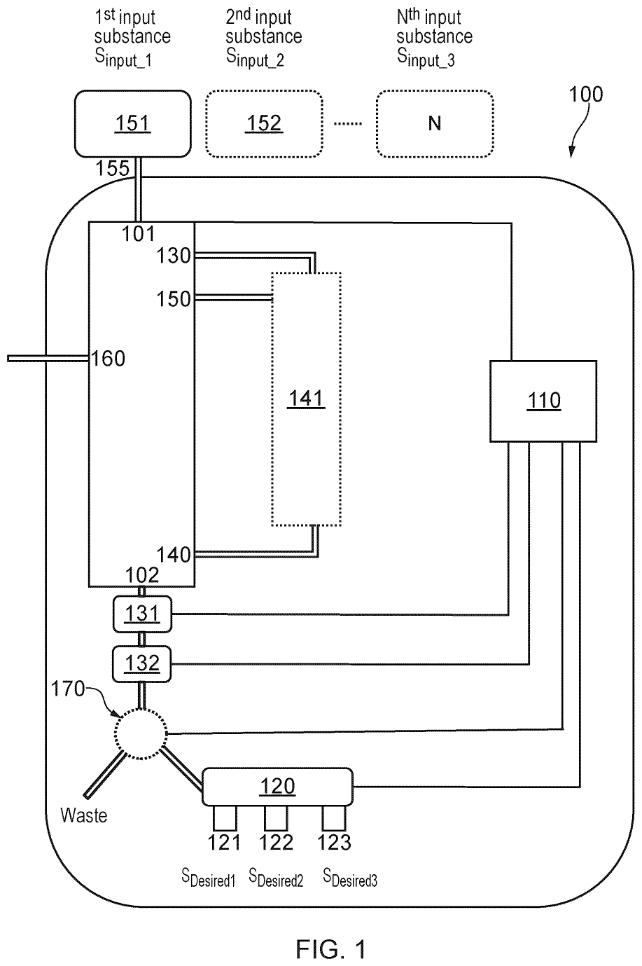

In the preset disclosure, the term “point of interest” (POI/POIS) may e.g., signify important events of a bioprocessing process (e.g. a chromatography process, such as beginning/end of chromatography run, beginning/end of elution, point of injection etc.). FIG. 1 shows a chromatography system 100 embodied as a chromatography apparatus according to one or more embodiments of the disclosure.

The chromatography system 100 is configured to provide a desired system functionality, typically to receive input substances S_{input_1}, S_{input_2}, S_{input_3} and produce one or more desired substances S_{Desired1}, S_{Desired2}, S_{Desired3}.

In one example, the chromatography system 100 comprises a chromatography apparatus configured to separate a desired substance or sample S_{Desired} from one or more input substances S_{Input1}-S_{InputN}, e.g., different mixtures of the sample and other compositions.

The chromatography system 100 may comprise a selection of bioprocessing units, such as a reservoirs 151, 152 . . . -N, a column 141, a splitter 170, at least one UV sensor, a pH sensor 131 and a conductivity sensor 132. The chromatography system 100 in the form of a chromatography apparatus is described in further detail below.

The chromatography apparatus 100 may typically comprise at least one inlet 155. The inlet may optionally be coupled to one or more reservoirs 151, 152 . . . -N configured to hold a fluid. It is understood that the chromatography apparatus 100 may comprise any number of reservoirs and corresponding inlets. The inlet 155 may e.g., be implemented as tubular elements such as a tube or hose. The chromatography apparatus 100 may further comprise a valve unit (not shown). The valve unit may be coupled to the reservoir(s) 151, 152 . . . -N by the inlet 155 coupled to the fluid inlet 101. The valve unit may be configured to be coupled to a (e.g. a first) column 141 by a first pair of fluid ports 130, 140. The first column 141 may be comprised in the chromatography apparatus 100 or arranged external to the chromatography apparatus 100.

The chromatography apparatus 100 may further comprise an intelligent packing fluid port or packing fluid port 150 configured to be coupled to a packing port of the column 141. The chromatography apparatus 100 may further comprise a waste fluid port 160 configured to be coupled to a waste reservoir or drain (not shown).

The chromatography apparatus 100 may further comprise or be operatively coupled to a control unit 110 which comprises circuitry, e.g., a processor and a memory. The memory may contain instructions executable by the processor, whereby said chromatography apparatus 100 is operative to perform any of the steps or methods described herein.

The chromatography apparatus 100 may optionally comprise a splitter 170 coupled to a selection of any of a pH sensor 131, a conductivity sensor 132 and an outlet valve 120. The splitter 170 may be configured to direct fluid to the outlet valve 120 or any other unit. Optionally the splitter 170 may be communicatively coupled to the control unit 110 and perform coupling of fluid in in response to a control signal from the control unit 110.

The pH sensor 131 may be communicatively coupled to the control unit 110 and configured for measuring the pH of the fluid provided by the splitter 170. One or more UV sensor(s) may also be provided to enable monitoring/detection of target protein products. The chromatography apparatus 100 may further comprise a conductivity sensor 132 communicatively coupled to the control unit 110 and configured for measuring the conductivity of the fluid provided by the splitter 170. The pH sensor 131 and/or the conductivity sensor 132 may further be configured to provide the measured pH and measured conductivity as control signals comprising measurement data to the control unit 110.

The chromatography apparatus 100 may further comprise an outlet valve 120 coupled to the splitter 170. The outlet valve 120 may have one or more outlets or outlet ports 121-123 and is configured to provide the fluid provided by the splitter 170 to the one or more outlets 121-123 in response to a control signal, e.g., received from the control unit 110.

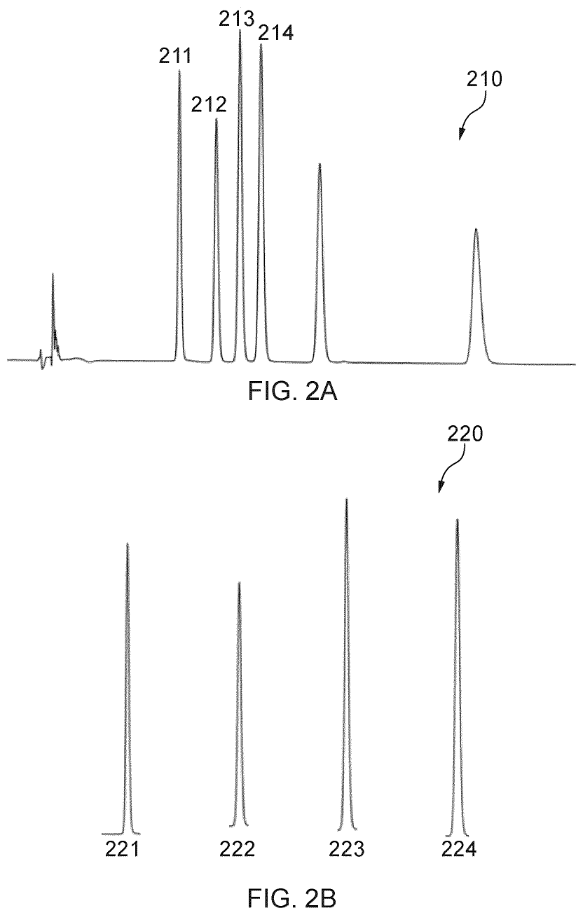

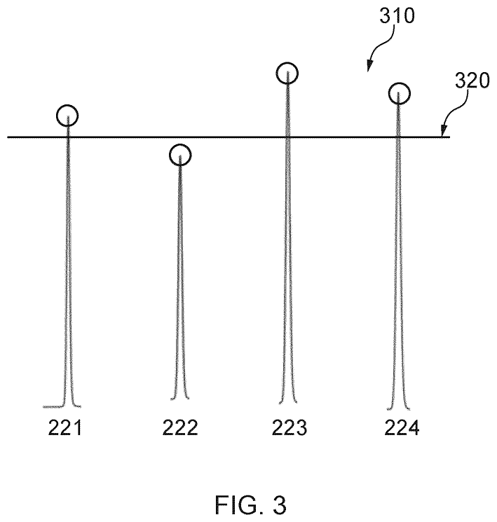

FIG. 2A illustrates an example of result data generated by one or more chromatography devices according to one or more embodiments of the disclosure. In this example, raw data in the form of a chromatogram 210 illustrating elution peaks 211-214 is generated during a chromatography run. For example, from a PCC cycle, a first peak may be generated during a wash and another peak during elution. However, it is noted that the techniques described herein are not only limited to peak-like data; e.g. it may be applied to analyse transitions, stable levels, etc.

FIG. 2B illustrates deriving of points of interest according to one or more embodiments of the disclosure. From the chromatogram 210 illustrated in FIG. 2A, a data source 220 derives points of interests 221-224 in the form of data surrounding respective of the elution peaks 211-214 from the chromatogram 210. Such points of interest 221-224 may be derived from marks in a run log provided at the start and end of interesting peaks, by finding all peaks and summing areas together, finding the largest peak or a peak delimited by retention restrictions, etc.

As can be seen from FIG. 2B, the derived points of interests 221-224 includes all data relevant for processing characteristics of elution peaks but comprises a significant reduction of the raw data.

This has the advantage that additional chromatography runs may be considered when analyzing result data. In other words, a better quality/statistical certainty of the analysis can be obtained by considering a larger set of data, e.g., when determining an elution peak.

In one example, a larger number of chromatography runs may be considered when comparing multiple chromatography runs for a particular sample. By comparing elution peaks from multiple chromatography runs, a better quality of an estimated characteristic of the elution peak can be obtained. Various comparisons may be made, for example: a same analysis for many runs may be performed and extracted values of each analysis compared with other analyses (e.g. the maximum height of peak 221 may be determined and a trend value derived from each run over those many runs or cycles in a run may be analysed with extracted values between cycles compared); or a comparison can be to make sure that peak height is within an interval, using trending values to see any decreased performance, or to determine if a peak is missing in any of the runs.

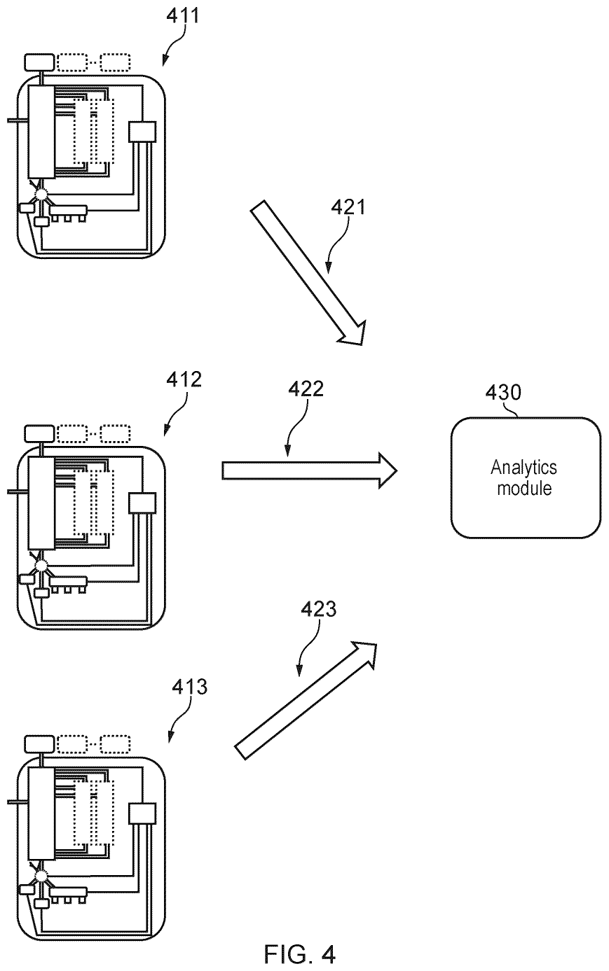

FIG. 3 illustrates an operation generating target data values according to one or more embodiments of the disclosure. With reference to FIG. 2A and FIG. 2B, a data source 310 may derive source data in the form of points of interest 221-224, e.g., in the form of elution peaks 211-214 that are derived from result data, such as a chromatogram 210. The maximum values of each elution peak 211-214 are illustrated with circles. An analytics module may execute a script comprising an operation defining an algorithm that generates the target data 320 using the source data.

In the example shown in FIG. 3, the operation receives the points of interest 221-224 in the form of elution peaks and generates an average peak value as target data 320.

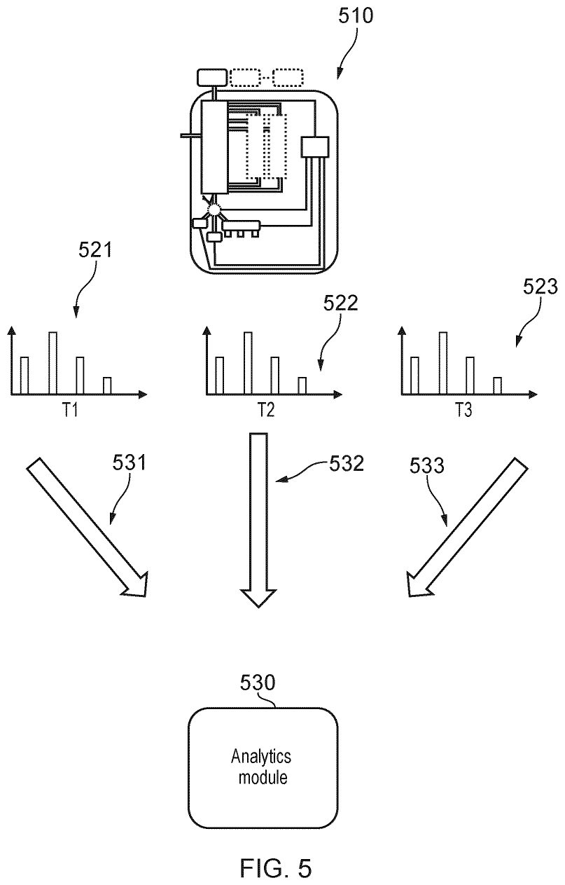

FIG. 4 illustrates raw result data generated from a plurality of chromatography systems according to one or more embodiments of the disclosure.

In one example, separation of identical samples is performed on three different chromatography systems 411-413.

Each of the chromatography systems 411-413 are provided with at least a data source 421-423 that are each configured to derive source data in the form of points of interests or subsets of data derived from the result data of the respective chromatography system 411-413. Source data from the data sources 421-423 can then be provided to an analytics module 430.

By analyzing result data from all of the chromatography systems 411-413, target data quality can be improved. In one example, an average of an elution peak may be calculated in the analysis. Various target data values 320 may be determined. These may then subsequently be used to help optimize protein product yield, to set boundaries to make sure a process works as intended, to measure lifetime parameters and see any decline/efficiency reduction in the process, etc. These may also be used for certain process analytics (PAT) requirements and be recorded over many production runs to use in obtaining and complying with various regulatory requirements.

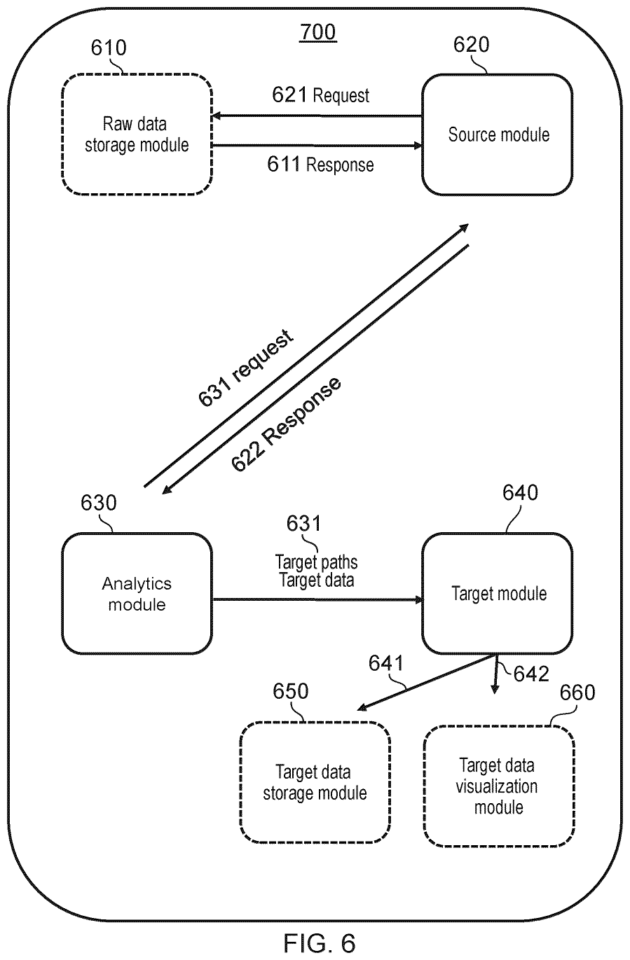

FIG. 5 illustrates raw result data 521-523 generated at different chromatography runs performed at different points in time according to one or more embodiments of the disclosure. In one example, a chromatograph system 100 subsequently performs chromatography runs of identical samples.

In one example, separation of identical samples is performed at three different points in time T1-T3 by the chromatography systems 510.

An identical data source 531-533 is provided for each chromatography run that is configured to derive source data in the form of points of interests or subsets of data derived from the result data of the chromatography system 510. Source data from the data sources 531-533 can then be provided to an analytics module 530.

By analyzing result data from all of the three different points in time T1-T3, target data quality can be improved. In one example, an average of an elution peak may be calculated in the analysis.

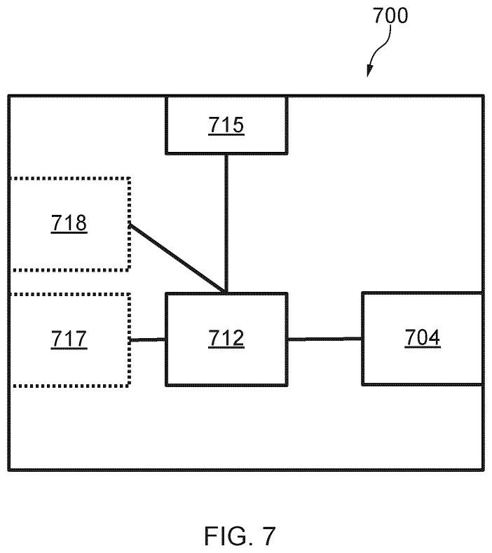

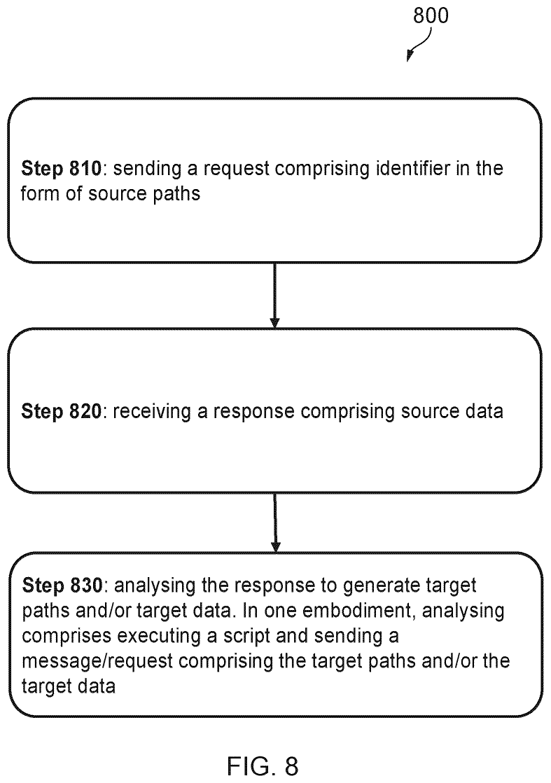

FIG. 6 illustrates flow of data between functional modules of a computer 700 performing a method according to one or more embodiments of the present disclosure. The computer is further described in relation to FIG. 7. The computer 700 is configured for configured to analyze source data/result data generated by one or more chromatography devices.

The computer comprises or is communicatively coupled to one or more functional modules.

A raw data storage module 610 is configured to store result data received from one or more chromatography systems 100 and provide stored data in response to requests.

A source module 620 is configured to derive source data and/or data sources from result data. The source data and/or data sources are typically indicative of points of interests or subsets of data derived from the result data. In one embodiment, the source module 620 is typically configured to receive one or more source paths in a request 631 from an analytics module 630. The source paths are identifiers identifying one or more raw data storage modules 610 comprising result data of interest. The source module 620 then resolves the one or more source paths. The source paths are identifying source data in the form of points of interests or subsets of data to be derived from the result data. The source module 620 then sends a request 621 to the one or more raw data storage modules 610. The one or more raw data storage modules 610 then sends a response 611 comprising the source data and/or points of interests and/or subsets of data of the result data to the source module 620. The source module 620 then sends a response comprising source data and/or data sources to the analytics module 630.

In various embodiments, a source path points out:
i) a resolver to act as integration code to resolve a path;
ii) a data path to point out a subset of data from the data source; and
iii) a parameter set acting as filter parameters to select specific values of ranges of data.

For example:

“Path”:aa://results/run/curve?run_id=${selected_run_id}&curve_name=$(curve_identi
fier}&retention_type=${retention_type}&segment=${segment)

is an example from a template: “${variables}” are values of template variables from the template, where “results” points to a resolver, “/run/curve” selects curve data, and the rest after “?” relates to parameters used by the results resolver to find the wanted curve data.

This has at least the advantage that the amount of data transferred is reduced. A further advantage is that physical storages of the result data are hidden from the analytics module 630. Any reconfiguration of physical storages can be made without notifying the analytics module 630.

Some base functionality for implementing embodiments of the invention may be provided by pre-existing software (e.g. in UNICORN™), which can provide a framework to load an analytical engine in. Certain embodiments of the present invention may then be provided as a platform extension (e.g. to UNICORN™) that will contain the analytical engine and base algorithms. Application specific extensions may also be provided.

The analytics module 630 is further configured to receive the response 622 comprising source data and/or data sources. The analytics module 630 is further configured to analyze the response 622 to generate target paths and target data, wherein analyzing comprises executing a script (see, for example, the example(s) as described below).

In one embodiment, the script comprises a selection of any of: instructions defining a flow of execution, operations defining an algorithm that generates the target data using the source data and/or rules defining if an operation should be executed or not using execution context. The analytics module 630 is further configured to send a message 631 comprising the target paths and/or the target data to a target module 640.

Examples of target data are baseline, Height Equivalent to a Theoretical Plate or HETP etc.

The target module 640 is configured to store and/or visualize target data. In one embodiment, the target module 640 is typically configured to receive one or more target paths in the message 631 from the analytics module 630. The target paths are identifiers identifying one or more target data storage modules and/or target data visualization modules 660. The target module 640 then resolves the one or more target paths. The target module 640 then optionally sends one or more messages 641, 642 to a target data storage module 650 configured to store target data and/or to a target data visualization module 660 configured to visualize the target data.

The sources and targets may comprise integration code. Sources can be created to read data from any data source including external databases and files. Targets can be a local database, visualization within a platform (e.g. UNICORN™) or any external target. Examples formats for these may be CSV, Excel file targets, etc. However, the specific data format is not a critical matter, since the source resolvers can translate complex datatypes into an information format that can be understood by an appropriate analysis engine.

FIG. 7 shows a computer 700 according to one or more embodiments of the present disclosure. The computer 700 may be in the form of e.g., a chromatography system, a computer, a server, an on-board computer, a stationary computing device, a laptop computer, a tablet computer, a handheld computer, a wrist-worn computer, a smart watch, a smartphone, or a smart TV. The computer 700 may comprise processing circuitry 712 communicatively coupled to a transceiver 704 configured for wired or wireless communication. The computer 700 may further comprise at least one optional antenna (not shown in figure). The antenna may be coupled to the transceiver 704 and is configured to transmit and/or emit and/or receive wired or wireless signals in a communication network, such as Wi-Fi, Bluetooth, 3G, 4G, 5G etc. In one example, the processing circuitry 712 may be any of a selection of a processor and/or a central processing unit and/or processor modules and/or multiple processors configured to cooperate with each-other. Further, the computer 700 may further comprise a memory 715. The memory 715 may e.g., comprise a selection of a hard RAM, disk drive, a flash drive or other removable or fixed media drive or any other suitable memory known in the art. The memory 715 may contain instructions executable by the processing circuitry to perform any of the steps or methods described herein. The processing circuitry 712 may be communicatively coupled to a selection of any of the transceiver 704 and the memory 715. The computer 700 may be configured to send/receive control signals directly to any of the above-mentioned units or to external nodes or to send/receive control signals via a wired and/or wireless communications network.

The wired/wireless transceiver 704 and/or a wired/wireless communications network adapter may be configured to send and/or receive data values or parameters as a signal to or from the processing circuitry 712 to or from other external nodes.

In an embodiment, the transceiver 704 communicates directly to external nodes or via a wireless communications network.

In one or more embodiments the computer 700 may further comprise an input device 717, configured to receive input or indications from a user and send a user input signal indicative of the user input or indications to the processing circuitry 712.

In one or more embodiments the computer 700 may further comprise a display 718 configured to receive a display signal indicative of rendered objects, such as text or graphical user input objects, from the processing circuitry 712 and to display the received signal as objects, such as text or graphical user input objects.

In one embodiment the display 718 is integrated with the user input device 717 and is configured to receive a display signal indicative of rendered objects, such as text or graphical user input objects, from the processing circuitry 712 and to display the received signal as objects, such as text or graphical user input objects, and/or configured to receive input or indications from a user and send a user-input signal indicative of the user input or indications to the processing circuitry 712.

In a further embodiment, the computer 700 may further comprise and/or be coupled to one or more additional sensors (not shown in the figure) configured to receive and/or obtain and/or measure physical properties pertaining to the computer and/or chromatography system and send one or more sensor signals indicative of the physical properties to the processing circuitry 712.

In one or more embodiments, the processing circuitry 712 is further communicatively coupled to the input device 717 and/or the display 718 and/or the additional sensors.

In embodiments, the communications network communicate using wired or wireless communication techniques that may include at least one of a Local Area Network (LAN), Metropolitan Area Network (MAN), Global System for Mobile Network (GSM), Enhanced Data GSM Environment (EDGE), Universal Mobile Telecommunications System, Long term evolution, High Speed Downlink Packet Access (HSDPA), Wideband Code Division Multiple Access (W-CDMA), Code Division Multiple Access (CDMA), Time Division Multiple Access (TDMA), Bluetooth®, Zigbee®, Wi-Fi, Voice over Internet Protocol (VoIP), LTE Advanced, IEEE802.16m, WirelessMAN-Advanced, Evolved High-Speed Packet Access (HSPA+), 3GPP Long Term Evolution (LTE), Mobile WIMAX (IEEE 802.16e), Ultra Mobile Broadband (UMB) (formerly Evolution-Data Optimized (EV-DO) Rev. C), Fast Low-latency Access with Seamless Handoff Orthogonal Frequency Division Multiplexing (Flash-OFDM), High Capacity Spatial Division Multiple Access (iBurst®) and Mobile Broadband Wireless Access (MBWA) (IEEE 802.20) systems, High Performance Radio Metropolitan Area Network (HIPERMAN), Beam-Division Multiple Access (BDMA), World Interoperability for Microwave Access (Wi-MAX) and ultrasonic communication, etc., but is not limited thereto.

Moreover, it is realized by the skilled person that the computer 700 may comprise the necessary communication capabilities in the form of e.g., functions, means, units, elements, etc., for performing the present solution. Examples of other such means, units, elements and functions are: processors, memory, buffers, control logic, encoders, decoders, rate matchers, de-rate matchers, mapping units, multipliers, decision units, selecting units, switches, interleavers, de-interleavers, modulators, demodulators, inputs, outputs, antennas, amplifiers, receiver units, transmitter units, DSPs, MSDs, TCM encoder, TCM decoder, power supply units, power feeders, communication interfaces, communication protocols, etc. which are suitably arranged together for performing the present solution.

Especially, the processing circuitry of the present disclosure may comprise one or more instances of a processor, processor modules and multiple processors configured to cooperate with each-other, Central Processing Unit (CPU), a processing unit, a processing circuit, a processor, an Application Specific Integrated Circuit (ASIC), a microprocessor, a Field-Programmable Gate Array (FPGA) or other processing logic that may interpret and execute instructions. The expression “processing circuitry” and/or “processing means” may thus represent a processing circuitry comprising a plurality of processing circuits, such as, e.g., any, some or all of the ones mentioned above. The processing means may further perform data processing functions for inputting, outputting, and processing of data comprising data buffering and device control functions, such as processing control, user interface control, or the like.

In one embodiment, a computer is provided, wherein the computer is configured to perform any or all of the method steps of the method described herein.

In one embodiment, a chromatography apparatus and/or system is provided, the chromatography apparatus and/or system comprising all or a selection of the features of the computer described in relation to FIG. 7. The chromatography apparatus or system is configured to perform any or all of the method steps of the method described herein.

In one embodiment, a computer program is provided comprising computer-executable instructions for causing a computer, when the computer-executable instructions are executed on a processing unit comprised in the computer, to perform any of the method steps of the method described herein.

In one embodiment, a computer program product is provided comprising a computer-readable storage medium, the computer-readable storage medium having the computer program above embodied therein.

In one embodiment, a carrier containing the computer program above is provided, wherein the carrier is one of an electronic signal, optical signal, radio signal, or computer readable storage medium.

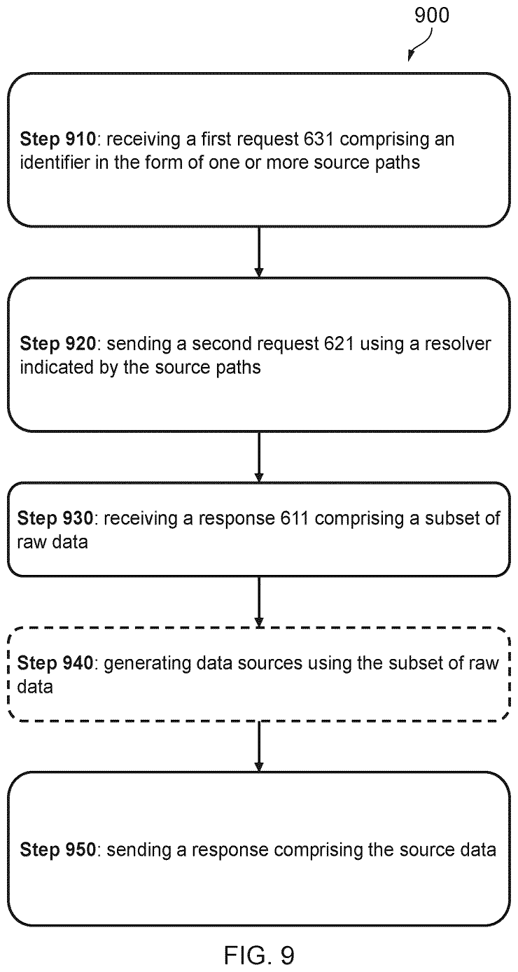

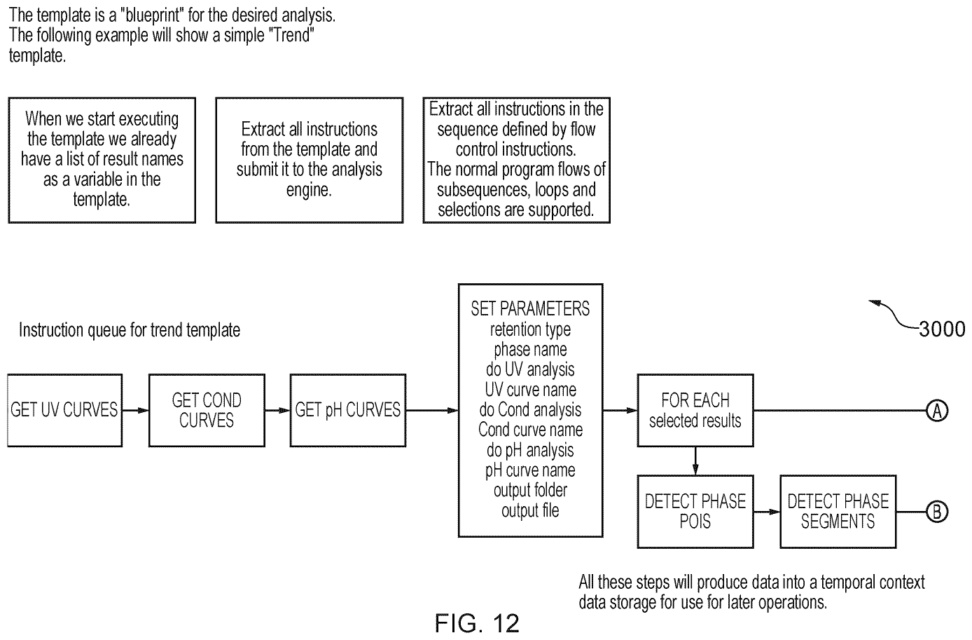

FIG. 8 shows a flowchart of a method 800 according to one or more embodiments of the present disclosure. The computer implemented method is performed by an analytics module 630 configured to analyze source data, e.g, generated by one or more chromatography devices. The method comprising:
Step 810: sending a request 631 comprising identifier in the form of source paths.
Step 820: receiving a response 622 comprising source data and/or data sources.
Step 830: analyzing the response 622 to generate target paths and/or target data. In one embodiment, analyzing comprises executing a script and sending a message/request 631 comprising the target paths and/or the target data. For example, analyzing of the response to generate target paths and/or target data may be implemented using an analysis engine configured as per FIG. 11, below.

In one embodiment, the source data and/or data sources are indicative of points of interests or subsets of data derived from the result data.

In one embodiment, the script comprises a selection of any of instructions defining a flow of execution, operations defining an algorithm that generates the target data using the source data and rules defining if an operation should be executed or not using execution context.

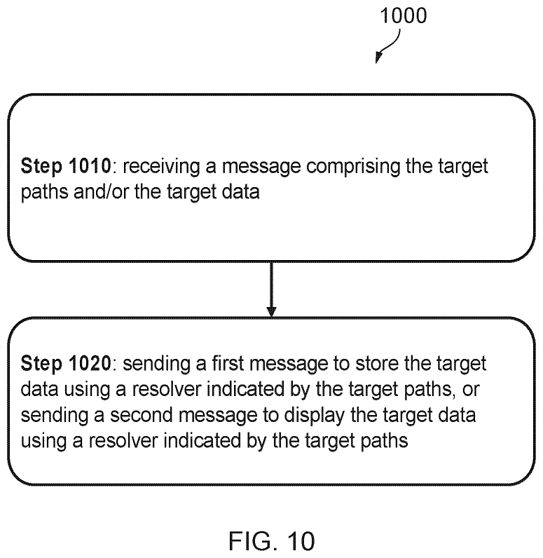

FIG. 9 shows a flowchart of a method 900 according to one or more embodiments of the present disclosure. The computer implemented method is performed by a source module 620 configured to generate source data and/or data sources using result data, e.g., generated by one or more chromatography devices. The method comprises:
Step 910: receiving a first request 631 comprising an identifier in the form of one or more source paths.
Step 920: sending a second request 621 using a resolver indicated by the source paths.
Step 930: receiving a response 611 comprising a subset of raw data.

Optional Step 940: generating source data and/or data sources using the subset of raw data. For example, a resolver may return a property packet of data selected from a data source and feed it into this operational step.
Step 950: sending a response 622 comprising the source data and/or data sources.

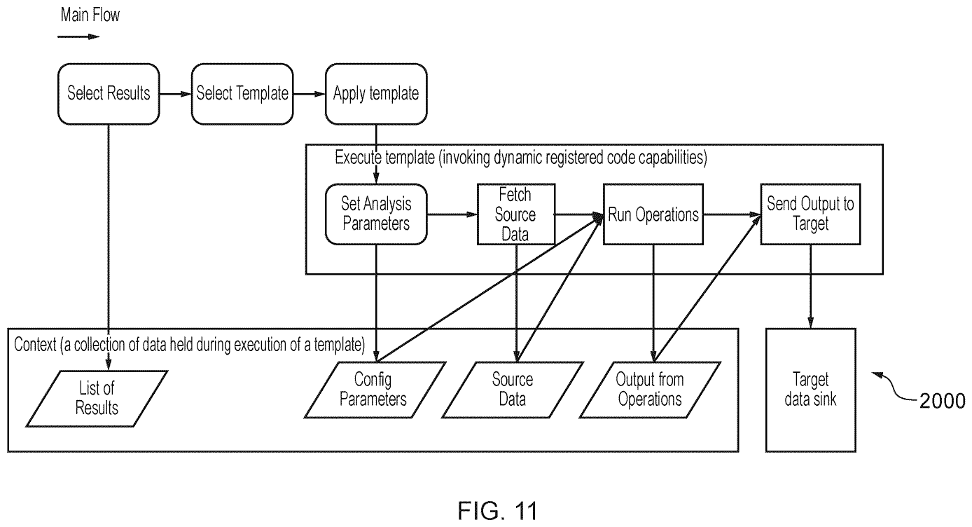

FIG. 10 shows a flowchart of a method 1000 according to one or more embodiments of the present disclosure. The computer implemented method is performed by a target module 640 configured to output target data using target paths. The method comprises:
Step 1010: receiving a message 631 comprising the target paths and/or the target data.
Step 1020: sending a first message 641 to store the target data using a resolver indicated by the target paths, or sending a second message 642 to display the target data using a resolver indicated by the target paths.

FIG. 11 shows a high-level practical workflow 2000 that may be used in various embodiments of the invention. For example, the workflow 2000 may be used by the analytics module 630 to analyze responses to generate target paths and target data.

Target paths may be set by a template script, that defines the workflow 2000, so that the analytics module 630 reading/executing those scripts knows where to put data, etc.

In the workflow 2000, which may be provided by an analysis engine, a user selects a number of results from a results browser. This is provided in this embodiment by UNICORN® software available from Cytiva® that is used for flexible control for chromatography, filtration, oligo synthesis and bioreactors. The results comprise source data generated by one or more such bioprocessing device 100 selected for subsequent analysis. Generally, such source data is indicative of how a measured bioprocessing data parameter evolves over time. For example, such data may depict bioprocessing data (y-axis) correlated with temporal data (x-axis). This may thus be presented as a set of peaks that show how the bioprocessing data varies with time (such as are depicted in FIGS. 2A, 2B and 3, for example).

These results are then passed to a context module that generates a collection of data that is held during execution of a template by the software.

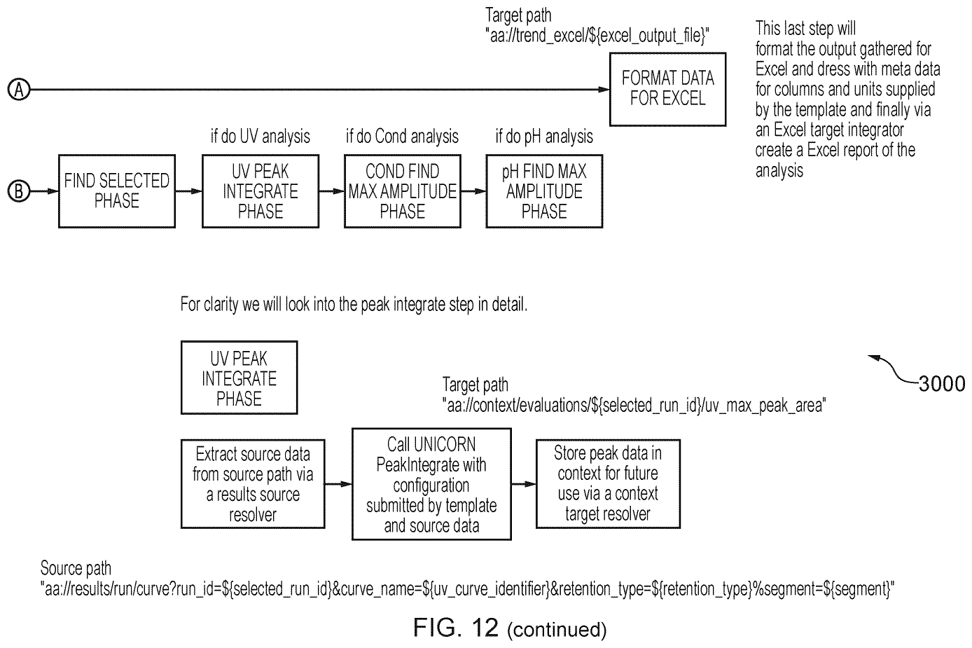

After selecting the results, the user may then also select a template using the advanced analytics tab of the UNICORN™ software which is then applied to the list of results (e.g. as is shown in FIG. 12). Execution of the template involves invoking dynamic registered code capabilities to determine target sink data corresponding to appropriate target paths and target data. This occurs by firstly setting analysis parameters which are also passed to the context module for later use. Data is then fetched by a fetch source data module which fetches the correct source data or meta data and transfers it to a run operations module, as well as copying it to the context module. The run operations module then uses the analysis the configuration parameters defined by the analysis parameters and the source/meta data to generate output from operations data corresponding to target sink data that is then passed to both the context module and an output target module.

The templates may be configured to prompt a user for further input as necessary, and code executed by the templates can be dynamically exchanged in UNICORN™. The output target module can also configure the output of the run operations into an appropriate format for further use, and transmits that data as required. For example, the data may be formatted in an Excel, ELN notebook, text file, etc. format.

FIG. 12 shows an example embodiment 3000 of template execution in more detail. In this example, the template is used to perform a relatively simple trend analysis (e.g. in the UNICORN™ software). In practice however, e.g. for a large dataset from a periodic counter-current chromatography (PCC) run, many thousands of instructions/modules may be required to perform appropriate analysis functions.

At the start of the template execution, a list of result names is provided as a variable therein. Instructions from the template are then extracted and submitted to the analysis engine. These are then all executed in sequence as defined by flow control instructions. The normal program flows of subsequences, loops and selections are supported.

In this instance, the instruction queue for the trend template comprises: i) get UV curves; ii) get conductivity curves; and iii) get pH curves. The SET PARAMETERS are: i) retention type; ii) phase name; iii) do UV analysis; iv) UV curve name; v) do conductivity (Cond) analysis; vi) Cond curve name; vii) do pH analysis; viii) pH curve name; ix) output folder; and x) output file.

In turn, and for each instruction in the instruction queue, data is fetched. A detect phase points of interest (POIS) is performed for each followed by detection of phase segments. Phases are a known concept in UNICORN™ and are denoted in the runlog, which may also be displayed in the Evaluation module thereof. The new functionality of embodiments of the present invention can use that information to find subsegments of data and read that from, e.g. a database, thereby limiting the amount of processing needed and allowing for a higher resolution of data.

For the UV analysis, a UV peak integration phase is performed. In this instance, source data is extracted from a source path via a results source resolver. The PeakIntegrate function of UNICORN™ is then called with a configuration submitted by the respective template and source data. Peak data is then stored in the context module for subsequent use via a context path resolver.

Conductivity analysis is also performed to find a maximum amplitude phase. The for the pH analysis, a pH find of the maximum amplitude phase is additionally determined.

The data determined from each instruction execution is also optionally formatted into an Excel format to enable reports to be generated for subsequent analysis.

Various embodiments of the present invention can thus provide a method, computer or program wherein source data, target data and/or raw data is generated by or provided to a bioprocessing device/system that comprises one or more of: a chromatography device, a cell culture device, a filtration device and/or an oligo synthesis device.

Such data can be indicative of how a bioprocessing-related magnitude value evolves over time (e.g. it may provide peaks of varying height/magnitude). Analysis of these may determine peak values/integrated area/averages etc. that can relate to physical time-variant bioprocessing parameters e.g. volume/concentration/etc. present during various phases of bioprocessing. Various embodiments of the present invention may thus be provided that mitigate or solve the drawbacks and problems described above in relation to conventional systems and devices by providing a more flexible way to select data and operations performed on said data.

Finally, it should be understood that the invention is not limited to the embodiments described above, but also relates to and incorporates all embodiments within the scope of the appended independent claims.

1. A computer implemented method performed by an analytics module configured to analyze source data generated from raw data generated by one or more bioprocessing device, the method comprising:
sending, to a source module, a request comprising an identifier in the form of source paths, the source module being configured to derive the source data from the raw data stored in one or more raw data storage modules, the source data being indicative of points of interests or subsets of data derived from the raw data,
receiving, from the source module, a response comprising the source data,
analyzing the response to generate target paths and target data, wherein analyzing comprises executing a script, the target paths being identifiers identifying one or more target data storage modules and/or target data visualization modules, and
sending, to a target module, a message comprising the target paths and the target data.

2. (canceled)

3. The method according to claim 1, wherein the script comprises a selection of any of instructions defining a flow of execution, operations defining an algorithm that generates the target data using the source data and rules defining if an operation should be executed or not using execution context.

4. A computer implemented method performed by a source module configured to generate source data generated from raw data generated by one or more bioprocessing device, the method comprising:
receiving, from an analytics module, a first request comprising an identifier in the form of source paths,
sending, to a raw data storage module, a second request using a resolver indicated by the source paths,
receiving, from the raw data storage module, a first response comprising a subset of raw data,
generating the source data using the subset of raw data, the source data being indicative of points of interests or subsets of data derived from the raw data, and
sending, to the analytics module, a second response comprising the source data.

5. A computer implemented method performed by a target module configured to output target bioprocessing data using target paths, the method comprising:
receiving, from an analytics module, a message comprising the target paths and the target data,
sending, to a target data storage module, a first message to store the target data using a resolver indicated by the target paths,
or
sending, to a target data visualization module, a second message to display the target data using a resolver indicated by the target paths.

6. A computer comprising:
a processor, and
a memory, said memory containing instructions executable by said processor, wherein said computer is configured to perform the method according to claim 1.

7. A computer program comprising computer-executable instructions for causing a computer, when the computer-executable instructions are executed on a processing unit comprised in the computer, to perform any of the method steps according to claim 1.

8. A computer program product comprising a computer-readable storage medium, the computer-readable storage medium having the computer program according to claim 7.

9. A method performed by a bioprocessing support system configured to analyze source data, the method comprising:
sending, from an analytics module to a source module, a first request comprising an identifier in the form of source paths,
sending, from the source module to a raw data storage module, a second request using a first resolver indicated by the source paths,
receiving, by the source module, a first response, from the raw data storage module, the first response comprising a subset of raw data, the raw data being generated by one or more bioprocessing devices,
generating, by the source module, source data using the subset of raw data, the source data being indicative of points of interests or subsets of data derived from the raw data,
sending, from the source module to the analytics module, a second response comprising the source data,
analyzing, by the analytics module, the second response to generate target paths and target data, wherein analyzing comprises executing a script, the target paths being identifiers identifying one or more target data storage modules and/or target data visualization modules, and
sending, from the analytics module to a target module, a first message comprising the target paths and the target data.

10. The method according to claim 9, the method further comprising:
sending, from the target module to a target data storage module, a second message to store the target data using a resolver indicated by the target paths, or
sending, from the target module to a target data visualization module, a third message to display the target data using a resolver indicated by the target paths.

11. The method of claim 1, wherein the bioprocessing device comprises one or more of: a chromatography device, a cell culture device, a filtration device and/or an oligo synthesis device.

12. The method of claim 11, wherein the source data, the target data and/or the raw data comprises bioprocessing data that denotes how a bioprocessing-related magnitude value evolves over time.
